(19) United States
(12) Patent Application Publication
(10) Pub. No.: US 2007/0161052 A1
(43) Pub. Date: Jul. 12, 2007

(54) TRPM5 based assays and the use thereof for the identification of modulators of sweet, bitter or umami (savory) taste

(75) Inventors: Guy Servant, San Diego, CA (US); Mark Williams, Carlsbad, CA (US); Andrew Patron, San Marcos, CA (US); Poonit Kamdar, San Diego, CA (US); Qing Chen, San Diego, CA (US); Bryan Moyer, San Diego, CA (US); David Dahan, Oceanside, CA (US); Tanya Ditschun, San Diego, CA (US); Sumita Ray, San Diego, CA (US); Amy Ligani, San Diego, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SENOMYX, INC., LaJolla, CA (US)

(21) Appl. No.: 11/583,086

(22) Filed: Oct. 19, 2006

Related U.S. Application Data
(60) Provisional application No. 60/727,832, filed on Oct. 19, 2005.

(51) Int. Cl. G01N 33/567

(52) U.S. Cl. 435/7.2

(57) ABSTRACT

Robust cell based assays are provided that use cells that express wild-type or modified TRPM5 nucleic acid sequences in order to identify putative taste modulators, preferably sweet, bitter and umami taste modulators. The preferred assays use HEK-293 cells that express TRPM5, optionally at least one GPCR, preferably a taste specific GPCR, and a G protein that couples therewith. These assays detect TRPM5 modulators by use of membrane potential dyes that emit fluorescence on changes in TRPM5 activity based on changes in membrane potential and these changes in fluorescence are detectable using Fluorimetric Imaging Plate Readers (FLIPR).

### RELATED APPLICATIONS

This application relates to and claims benefit of priority to U.S. provisional application Ser. No. 60/727,832 filed on Oct. 19, 2005. This application is incorporated by reference in its entirety herein.

### FIELD OF THE INVENTION

The present invention relates to the use of rodent and human TRPM5 nucleic acid sequences and the polypeptides encoded thereby in assays for identifying modulators of taste, preferably bitter, sweet or umami (savory) taste. This invention more particularly provides cell-based assays using cells that express murine or human TRPM5 for screening for compounds that modulate TRPM5 activity and which modulate bitter, sweet or umami taste as confirmed in animal or human taste tests.

### BACKGROUND OF THE INVENTION

This invention relates to the use of wild-type and modified TRPM5 encoding nucleic acid sequences for identifying novel TRPM5 modulatory compounds, preferably compounds that modulate different (sweet, bitter and umami) taste modalities. TRPM5 encodes a member of the TRP receptor family (that is activated by calcium cations) (Prawitt et al., PNAS 100(25):15160-5 (epub 2003); Hoffman et al., Curr Biol. 13(13):1153-8 (2003)). Rodent and human TRPM5 sequences are known in the art. Additionally, it has been suggested that this ion channel is involved in bitter, sweet, and umami taste recognition. (Zhang et al., Cell 112:293-301 (2003); Amrein et al., Cell 112(3):283-4 (2003); Perez et al., Cell Calcium 33:541-9 (2003)).

TRPM5 is expressed on the surface of taste receptor cells and is activated by an elevation of intracellular Ca++ ions. This ion channel is a non-selective channel that is equally permeable to Na+, Cs+, or K+ but which is impermeable to Ca++.

However, notwithstanding what has been reported, improved assays and test kits for identifying compounds that modulate the human TRPM5 channel are needed. In particular, cell-based assays which utilize cells that efficiently express functional human TRPM5 which may be used to screen for compounds that modulate the human TRPM5 channel and which modulate specific taste modalities in humans and other mammals are needed. These compounds have application in foods, beverages, medicinals, cosmetics, and other compositions for oral administration.

### OBJECTS OF THE INVENTION

It is an object of the invention to provide novel assays using cells that express rodent or human TRPM5 nucleic acid sequences, preferably human cells, and which yield a TRPM5 ion channel polypeptide suitable for identifying TRPM5 modulatory compounds, e.g., novel taste modulators.

Also, it is an object of the invention to provide novel human TRPM5 nucleic acid sequences which comprise mutations relative to the native (wild-type) human TRPM5 nucleic acid sequence which are intended to optimize expression in specific recombinant host cells, preferably mammalian, and most preferably primate, e.g., human cells.

Particularly, it is an object of the invention to provide a modified human TRPM5 nucleic acid sequence, i.e., which possesses a different nucleic acid sequence than the previously reported naturally occurring human TRPM5 nucleic acid sequence, wherein such modified sequence contains mutations that are engineered in order to optimize TRPM5 expression in human cells and further wherein such mutations do not substantially alter the binding and/or functional properties of the resultant human TRPM5 polypeptide. Preferably, these mutations will not alter the native human TRPM5 polypeptide sequence i.e. will be silent or will not appreciably alter the TRPM5 polypeptide sequence, e.g., conservative amino acid substitutions. For example, such mutations may remove one or more of the following: (i) putative human putative internal TATA-boxes, (ii) chi-sites, (iii) ribosomal entry sites, (iii) AT-rich or GC-rich sequence stretches, (iv) ARE, INS or CRS sequence elements and (v) cryptic splice donor and acceptor sites. Additionally, such mutations may replace one or more codons with host cell preferred codons, particularly human preferred codons.

Still more preferably, it is an object of the invention to provide the mutated human TRPM5 nucleic acid sequence contained in SEQ ID NO: 2.

Preferably, the subject TRPM5 nucleic acid sequences will be inserted into recombinant cells for use in cell-based assays that monitor TRPM5 activity using membrane potential dyes such as Na+, K+, Li+, or Cs+ sensitive membrane potential or fluorescent dyes.

In another embodiment, the changes in electrical activity by TRPM5 may be measured an ion flux assay, e.g., using a radiolabeled ion flux assay.

In another embodiment, TRPM5 activity may be monitored by ion flux assays that detect ion flux by atomic absorption spectroscopy.

In another embodiment the subject TRPM5 nucleic acid sequences may be inserted into recombinant cells for use in cell-based assays that monitor TRPM5 activity by electrophysiological methods, e.g., oocytes by patch clamping or two electrode voltage clamping techniques.

Most preferably, cells that express the subject TRPM5 nucleic acid sequences will be incorporated in cell-based assays that use a high-throughput screening platform which facilitates the screening of thousands or even millions of different putative taste modulatory compounds for the identification of TRPM5 modulators. Preferably these cells will also express at least one taste receptor such as a T1R or T2R taste receptor, preferably a human or rodent T1R or T2R. The sequences of such T1Rs and T2Rs are known in the art and are contained in numerous patent applications assigned to Senomyx and to the University of California naming Charles Zuker as an inventor. Additionally, these cells will typically be engineered to express or the cell will endogenously express a G protein such as a promiscuous G protein such as Galpha15, Galpha16, gustducin, transducin, another G protein or a chimera thereof. The TRPM5, taste GPCR, and G protein nucleic acid sequences may be comprised on the same or different vectors, e.g., plasmids.

Also, the subject TRPM5 nucleic acid sequences and cells containing may be incorporated in test kits useful for identifying compounds that modulate rodent or human TRPM5 and specific taste modalities which comprise (i) a test cell that expresses a native or an altered human TRPM5 nucleic acid sequence according to the invention and (ii) a detection system that comprises a means for measuring TRPM5 activity, e.g., fluorimetric or electrophysiological means for identifying compounds that modulate the activity of human TRPM5.

### BRIEF DESCRIPTION OF THE INVENTION

The present invention relates to robust cell-based screening assays for identifying compounds that modulate TRPM5 function and which modulate specific taste modalities as confirmed in human and animal taste tests.

Also, the present invention also relates to novel mutated TRPM5 nucleic acid sequences which contain mutations engineered in order to optimize expression in desired host cells and the use of these host cells for identifying human TRPM5 modulatory compounds, preferably compounds that function as taste modulators themselves and/or which enhance the effect of other tastants, e.g., bitter, sweet or savory tasting compounds.

As noted previously, TRPM5 is a non-selective cation channel in the TRP ion channel family that is expressed on the surface of taste receptor cells that is believed to be significant for the recognition of bitter, sweet, and umami taste modalities in mammals. This channel is equally permeable to Na+, Cs+, and K+ but is impermeable to Ca++.

Based on these properties the present inventors sought to develop novel and robust screening assays using cells that transiently or stably express rodent or human TRPM5 nucleic acid sequences, preferably human cells, and more preferably HEK293 cells in order to screen for taste modulatory compounds.

More particularly, the invention relates to assays that use HEK-293 cells that stably or transiently express TRPM5 to screen the effect of different putative TRPM5 modulatory compounds on TRPM5 activity using membrane potential dyes wherein changes in TRPM5 activity are detected fluorimetrically using a Fluorimetric Imaging Plate Reader (FLIPR). Compounds that affect TRPM5 activity have potential application as taste modulators which may be confirmed in human or animal taste tests

Particularly the invention provides novel and robust screening assays using hTRPM5 and mouse TRPM5 transiently or stably expressed in HEK-293 cells, membrane potential dyes and a Fluorimetric Imaging Plate Reader (FLIPR). It is demonstrated herein that the activation of GPCRs to phospholipase C and Ca++ mobilization (via either Gq or promiscuous proteins such as Galpha15) in cells that express mouse or human TRPM5 lead to significant changes in membrane potential, as revealed by a drastic increase in the fluorescence of the membrane potential dye which does not occur in the absence of the TRPM5 sequence.

In the case of assays which use human TRPM5, these assays may use cells which stably express or transiently express the wild-type TRPM5 or may use cells which express a modified TRPM5 sequence that is modified to enhance expression in human cells by the removal of at least one of (i) putative internal TATA boxes, (ii) chi-sites and ribosomal entry sites, (iii) AT-rich or GC-rich sequence stretches, (iv) ARE, INS, and CRS sequence elements and (v) cryptic splice donor and acceptor sites. Ideally, and in the exemplified modified human TRPM5 nucleic acid sequence these modifications do not affect the amino acid sequence of the resultant TRPM5 polypeptide. The exemplified modified human TRPM5 nucleic acid sequence is only 77% identical to the wild-type human TRPM5 nucleic acid sequence and contains no non-silent modifications. As noted, these cells may additionally express a GPCR such as a taste GPCR e.g., a human or rodent T1R or T2R, and a G protein such as a promiscuous G protein such as Galpha15 or a Gq protein.

As shown in the exemplified assays, the wild-type and mutated TRPM5 genes produced indistinguishable responses. The use of specific calcium ionophores such as ionomycin or A-23187 in human TRPM5 and mouse TRPM5 expressing HEK293 cells also lead to TRPM5-dependent changes in membrane potential, as revealed by a drastic increase in the fluorescence of the membrane potential dye. The observed effects are dependent on the presence of an appropriate cation, i.e., in the examples extracellular Na+. However, the invention encompasses other assay conditions such as the replacement of Na+ with another monovalent cation such as K+, Cs+, Li+, or another monovalent cation that permeates the TRPM5 ion channel.

Alternatively, the invention embraces assays wherein, human and rodent TRPM5 activity is detected using specific Na+ and K+ fluorescent dyes such as SBFI and PBFI, using radiolabeled Na+ flux assays or non-radiolabeled Li+ or Rb+flux assays coupled to atomic absorption spectroscopy as well as classical patch clamp and voltage clamp assays.

Surprisingly, it was found that the stimulation of TRPM5 with sub-optimal concentration of a GPCR agonist (leading to at most 25% activity of TRPM5) is optimal for the detection of enhancer compounds. Using the inventive robust assays a chemical library of about 200,000 compounds was screened and this screening resulted in the identification of several compounds that modulated the activity of human TRPM5 (inhibit or enhance the activity thereof). In human taste tests one of these modulators was found to significantly enhance the sweetness intensity of a Fructose/Glucose solution. Based on these and other results disclosed herein, it is anticipated that the subject TRPM5 assays will identify compounds which themselves, or derivatives thereof or compounds that are structurally related thereto will be useful as flavor additives for modulating the taste (bitter, sweet, or umami) of various consumer products.

Based thereon, cells which transiently or stably express TRPM5 and preferably at least one GPCR such as a taste receptor GPCR, e.g., a T1R or T2R, are potentially useful in screens, e.g., high-throughput platform screens to identify and quantify the effects of TRPM5 modulators which have potential as taste modulatory compounds

As noted above, the present invention also relates to mutated or altered human TRPM5 nucleic acid sequences which mutated to optimize expression in specific mammalian cells, preferably primate, most preferably human cells, relative to the wild-type human TRPM5 genomic or cDNA sequence (contained in SEQ ID NO:1 infra). Such optimized TRPM5 sequences will preferably retain the identical amino acid sequence as the wild-type human TRPM5 polypeptide or will only comprise inconsequential modifications. For example, the modified sequence will typically possess at least 85% sequence identity to native human TRPM5 polypeptide, more preferably at least 95% sequence identity therewith, and still more preferably at least 96-99% sequence identity therewith and will substantially retain the same binding and/or functional properties as the native human TRPM5 ion channel.

The present invention exemplifies a modified human TRPM5 nucleic acid sequence which encodes a polypeptide that is identical to the native human TRPM5 polypeptide having the sequence contained in SEQ ID NO. 2. This modified TRPM5 nucleic acid sequence has been engineered to optimize expression by the removal of putative internal TATA-boxes, chi-sites and ribosomal entry sites; AT-rich and GC-rich sequence stretches, ARE, INS and CRS sequence elements and cryptic splice donor and acceptor sites. The exemplified modified human TRPM5 nucleic acid sequence contains 808 silent nucleotide substitution mutations and exhibits about 77% nucleotide sequence identity to the reported native human TRPM5 nucleic acid sequence contained in SEQ ID NO: 1 infra. Cell-based assays using this modified human TRPM5 sequence are capable of identifying compounds that function as taste modulators in humans and other mammals.

### BRIEF DESCRIPTION OF THE FIGURES

FIG. 1 contains a sequence alignment of a modified (“optimized”) hTRPM5 sequence according to the invention and the previously reported wild-type hTRPM5 sequence. The native sequence is contained in SEQ ID NO: 1 and the altered sequence in SEQ ID NO:2.

FIG. 2 contains the protein sequence of human TRPM5.

FIG. 3 contains an experiment that assayed TRPM5 responses in cells contacted with the Galphaq-coupled receptor agonist carbachol.

FIG. 4 contains an experiment that assayed TRPM5 responses in cells contacted with the Galphaq-coupled receptor agonists carbachol, angiotensin II and histamine.

FIG. 5 contains an experiment using HEK-293 cells that express the H1 histamine receptor alone or that co-express the H1 histamine receptor and hTRPM5 that reveals that receptor-induced changes in membrane potential are hTRPM5-dependent.

FIG. 6 contains the results of an experiment using cells transfected with plasmids encoding the H1 histamine receptor and hTRPM5 which reveals that Galphaq-coupled receptor-induced changes in membrane potential require the presence of a monovalent cation (Na+).

FIG. 7 contains an experiment that assayed human TRPM5 responses to the Ca++ ionophore ionomycin in HEK-293 cells transfected with a plasmid encoding hTRPM5.

FIG. 8 contains an experiment that monitored mTRPM5 responses to the calcium ionophore ionomycin and Galphaq-coupled receptor agonists in HEK-293 cells transfected with a plasmid encoding mTRPM5.

FIG. 9 contains an experiment that monitored hTRPM5 and mTRPM5 responses to a Galpha15-coupled receptor agonist in cells stably expressing a bitter taste receptor (mT2R05 which responds to cycloheximide), Galpha15, and mouse TRPM5 or a control plasmid (pUC).

FIG. 10 contains an experiment that measured hTRPM5 response in a stable HEK-293 clone expressing hTRPM5 in pcDNA3.Neo in response to carbachol.

FIG. 11 contains the results of a TRPM5 modulator screening assay that screened a library of 15,000 compounds against HEK-293 cells that express hTRPM5 and which were stimulated with carbachol.

FIG. 12 contains a listing of hTRPM5 enhancers discovered in the screening assay depicted in FIG. 11.

FIG. 13 contains an experiment that reveals that hTRPM5 enhancers improve the potency and efficacy of carbachol-induced changes in membrane potential.

FIG. 14 contains an experiment which revealed that hTRPM5 enhancer compounds improve the potency and efficacy of ionomycin-induced changes in membrane potential.

FIG. 15 contains an experiment that compares the enhancer properties of an enhancer compound at different levels of TRPM5 activity and at different concentrations of enhancer in the presence of carbachol.

FIG. 16 contains an experiment that compares the enhancer properties of another TRPM5 enhancer compound at different levels of hTRPM5 activity and at different concentrations of enhancer in the presence of carbachol.

FIG. 17 compares the enhancer properties of another hTRPM5 enhancer compound at different levels of hTRPM5 activity and at different concentrations of enhancer in the presence of carbachol.

FIG. 18 contains the results of a FLIPR assay which revealed that the wild-type and mutated hTRPM5 sequences yielded identical results

FIG. 19 shows the activity distribution of 100,000 compounds screened in the TRPM5 assays according to the invention.

FIG. 20 shows that a hTRPM5 enhancer compound identified using the subject assays is a potent hTRPM5 enhancer with ideal in vitro properties.

FIG. 21 confirms the results of the experiment contained in FIG. 20 using patch clamp experiments.

FIG. 22 contains the results of experiments that determined the enhancer properties of the same enhancer compound in FIGS. 20 and 21 in a taste test.

FIG. 23 shows the discovery of a potent human TRPM5 blocker with ideal in vitro properties.

### Detailed Description of the Invention and Definition of Relevant Terms

The present invention provides assays that use wild-type or modified human or rodent TRPM5 nucleic acid sequences that have been “optimized” in order to provide for efficient expression in recombinant host cells, preferably mammalian cells, more preferably human cells and oocytes and assays using these TRPM5 expressing cells to identify TRPM5 modulators.

More specifically the invention relates to the subject modified human TRPM5 nucleic acid sequences and their expression as active channels in recombinant host cells, preferably human cells and more preferably HEK-293 host cells and cell-based assays using these cells.

As noted above, TRPM5 proteins form channels that are activated by an elevation in intracellular calcium. The TRPM5 protein has little selectivity among monovalent cations. Channel activity and changes therein in response to TRPM5 modulatory compounds therefore can be effectively measured, e.g., by recording changes in TRPM5 activity based on changes in membrane potential fluorimetrically using a variety of different ion and membrane potential dyes.

Preferably the effect of a putative TRPM5 modulator on TRPM5 activity is assayed using a novel and robust screening assay exemplified herein that uses HEK-293 cells that stably or transiently human or rodent TRPM5 and which further loads these cells with a suitable membrane potential dye and which measures changes in fluorescence automatically, e.g. by use of a Fluorimetric Imaging Plate Reader (FLIPR). These cells also will preferably express a GPCR e.g., a taste GPCR such as a T1R or a T2R.

As shown infra the activation of G-coupled receptors such as T1Rs and T2Rs coupling to phospholipase C and Ca++ mobilization (via either Galphaq or promiscuous G proteins such as Galpha15) in mouse or human TRPM5 expressing HEK-293 cells results insignificant changes in membrane potential, as revealed by a drastic increase in the fluorescence of the membrane potential dye whereas no comparable increase in membrane potential is observed in the absence of TRPM5 during GPCR stimulation.

As shown infra, in the exemplified assays the results are dependent on the presence of extracellular Na+ during the assay. However, it is anticipated that this monovalent cation may be replaced with other monovalent cations such as K+, Li+, Cs+ and the like.

Alternatively, TRPM5 activity can be detected using specific Na+ or K+ or other ion specific fluorescent dyes such as SBFI and PBFI.

Still alternatively, TRPM5 activity can be detected by using radiolabeled Na+ flux assays or non-radiolabeled Li+ or Rb+flux assays coupled to atomic spectroscopy.

Yet alternatively, TRPM5 activity can be detected by the use of classical patch clamp and voltage clamp experiments

Therefore, related to the foregoing, the invention provides assays useful for screening for modulators, e.g., activators, inhibitors, stimulators, enhancers, etc., of TRPM5 using the subject wild-type or modified TRPM5 nucleic acid sequences which are engineered in order to optimize expression in human cells that modulate taste. Such modulators can affect TRPM5 activity, e.g., by modulating TRPM5 transcription, translation, mRNA or protein stability; by altering the interaction of TRPM5 with the plasma membrane, or other molecules; or by affecting TRPM5 protein activity. Compounds are screened, e.g., preferably using high throughput screening (HTS), to identify those compounds that can bind to and/or modulate the activity of a TRPM5 polypeptide.

As disclosed herein, TRPM5 proteins are recombinantly expressed in cells, e.g., human cells which are transfected or transformed with a wild-type or an optimized human TRPM5 nucleic acid sequence according to the invention, and the modulation of TRPM5 is assayed by using any measure of ion channel function, such as measurement of the membrane potential, or measures of changes of TRPM5 activity using fluorescent dyes such as Na+ and K+ fluorescent dyes. A human TRPM5 agonist identified as set forth in the current application can be used for a number of different purposes. For example, a TRPM5 activator can be included as a taste modulator in foods, beverages, medicinals, or cosmetic compositions. For example such compounds may block bitter taste or enhance savory or sweet taste.

The present invention further provides kits for carrying out the herein-disclosed assay methods.

In order to further explain the invention the following definitions of certain terms are provided. In all other instances terms and phrases used in this application are to be given their ordinary meaning as they would be construed by one skilled in the art.

Definitions

“Cation channels” are a diverse group of proteins that regulate the flow of cations across cellular membranes. The ability of a specific cation channel to transport particular cations typically varies with the valency of the cations, as well as the specificity of the given channel for a particular cation.

“Homomeric channel” refers to a cation channel composed of identical alpha subunits, whereas “heteromeric channel” refers to a cation channel composed of two or more different types of alpha subunits. Both homomeric and heteromeric channels can include auxiliary beta subunits.

A “beta subunit” is a polypeptide monomer that is an auxiliary subunit of a cation channel composed of alpha subunits; however, beta subunits alone cannot form a channel (see, e.g., U.S. Pat. No. 5,776,734). Beta subunits are known, for example, to increase the number of channels by helping the alpha subunits reach the cell surface, change activation kinetics, and change the sensitivity of natural ligands binding to the channels. Beta subunits can be outside of the pore region and associated with alpha subunits comprising the pore region. They can also contribute to the external mouth of the pore region.

The term “authentic” or “wild-type” or “native” human “TRPM5” protein herein refers to the polypeptide encoded by the human TRPM5 nucleic acid sequence contained in SEQ ID NO:1 infra.

The term “authentic” or “wild-type” or “native” human TRPM5 nucleic acid sequence herein refers to the nucleic acid sequence contained in SEQ ID NO:1 infra.

The term “modified” or “optimized” human TRPM5 nucleic acid sequence according to the invention refers to a human TRPM5 nucleic acid sequence which has been modified or altered relative to the wild-type or authentic human TRPM5 nucleic acid sequence in order to optimize expression in desired host cells, preferably human cells or oocytes. In particular, such modified sequences are modified relative to authentic TRPM5 nucleic acid sequence by at least one of the following:

(i) removal of one or more putative TATA-boxes, chi-sites and ribosomal entry sites;

(ii) removal of one or more AT-rich or GC-rich stretches, ARE, INS or CRS sequence elements;

(iii) removal of one or more cryptic splice donor or acceptor sites; and

(iv) substitution of one or more codons with host preferred codons, in particular human preferred codons.

Typically, the subject modified TRPM5 segment will comprise at least 100 silent mutations, more typically at least 200-400, or even more typically at least about 400-800 silent mutations.

In the exemplified embodiment the modified human TRPM5 nucleic acid sequence will comprise the nucleic acid sequence contained in SEQ ID NO:2 which comprises over 800 silent mutations (mutations which do not affect polypeptide sequence relative to authentic human TRPM5 polypeptide encoded by SEQ ID. NO:1). This nucleic acid sequence possesses about 77% sequence identity with the nucleic acid sequence contained in SEQ ID NO:1. However, as noted previously the subject invention also contemplates modified human TRPM5 nucleic acid sequences which are modified as described to optimize expression which contain mutations which are not silent provided that these modifications do not appreciably impact the ligand binding and functional properties of the resultant human TRPM5 polypeptide vis-à-vis native human TRPM5 polypeptide. For example, such modifications may include one or more conservative amino acid substitution mutations. Preferably, an optimized or altered human TRPM5 nucleic acid sequence according to the invention will possess at most 90% sequence identity to the sequence in SEQ ID NO:1, more preferably at most 85-88% and still more preferably less than 85%, i.e. 75-85% sequence identity with SEQ ID NO:1.

Generically, a nucleic acid encoding any “TRPM5” or a fragment thereof (i.e., wherein TRPM5 is not limited to human TRPM5) refers to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to an amino acid sequence encoded by a TRPM5 nucleic acid or amino acid sequence of a TRPM5 protein, e.g., the sequence encoded by SEQ ID NO:1; (2) specifically bind to antibodies, e.g., polyclonal antibodies, raised against an immunogen comprising an amino acid sequence of a TRPM5 protein or immunogenic fragments thereof, and conservatively modified variants thereof; (3) specifically hybridize under stringent hybridization conditions to an anti-sense strand corresponding to a nucleic acid sequence (SEQ ID NO:1) encoding a TRPM5 protein, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to a TRPM5 nucleic acid, e.g., SEQ ID NO:1 or a rat or murine TRPM5 nucleic acid sequence. The nucleic acid and amino acid sequences for rat and murine TRPM5 are known.

A TRPM5 polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. These channels are all non-specific TRP channels which are permeable to Na+, Cs+ and K+ but impermeable to Ca++.

By “determining the functional effect” or “determining the effect on the cell” is meant assaying the effect of a compound that increases or decreases a parameter that is indirectly or directly under the influence of a TRPM5 polypeptide e.g., functional, physical, phenotypic, and chemical effects. Such functional effects include, but are not limited to, changes in ion flux, membrane potential, current amplitude, and voltage gating, a as well as other biological effects such as changes in gene expression of TRPM5 or of any marker genes, and the like. The ion flux can include any ion that passes through the channel, e.g., sodium, potassium, or cesium and analogs thereof such as radioisotopes. Such functional effects can be measured by any means known to those skilled in the art, e.g., patch clamping, using voltage-sensitive dyes, or by measuring changes in parameters such as spectroscopic characteristics (e.g., fluorescence, absorbance, refractive index), hydrodynamic (e.g., shape), chromatographic, or solubility properties.

In the present invention “functional effect” is preferably determined by changes in membrane potential detected by fluorimetric imaging or electrophysiologically, e.g., by patch clamp or two electrode voltage techniques.

“Inhibitors,” “activators,” and “modulators” of TRPM5 polynucleotide and polypeptide sequences are used to refer to activating, inhibitory, or modulating molecules identified using in vitro and in vivo assays of TRPM5 polynucleotide and polypeptide sequences. Inhibitors are compounds that, e.g., bind to, partially or totally block activity, decrease, prevent, delay activation, inactivate, desensitize, or down regulate the activity or expression of TRPM5 proteins, e.g., antagonists. “Activators” are compounds that increase, open, activate, facilitate, enhance activation, sensitize, agonize, or up regulate TRPM5 protein activity. Inhibitors, activators, or modulators also include genetically modified versions of TRPM5 proteins, e.g., versions with altered activity, as well as naturally occurring and synthetic ligands, antagonists, agonists, peptides, cyclic peptides, nucleic acids, antibodies, antisense molecules, siRNA, ribozymes, small organic molecules and the like. Such assays for inhibitors and activators include, e.g., expressing TRPM5 protein in vitro, in cells, cell extracts, or cell membranes, applying putative modulator compounds, and then determining the functional effects on activity, as described above.

Samples or assays comprising TRPM5 proteins that are treated with a potential activator, inhibitor, or modulator are compared to control samples without the inhibitor, activator, or modulator to examine the extent of activation or migration modulation. Control samples (untreated with inhibitors) are assigned a relative protein activity value of 100%. Inhibition of TRPM5 is achieved when the activity value relative to the control is about 80%, preferably 50%, more preferably 25-0%. Activation of TRPM5 is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, siRNA, oligonucleotide, ribozyme, etc., to be tested for the capacity to modulate taste sensation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequences SEQ ID NO:2), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nucl. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma.-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (.sub.3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., transmembrane domains, pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include 32ₚ, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (T.m) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formarnmide, 5.times. SSC, and 1% SDS, incubating at 42° C., or, 5×SSC, 1% SDS, incubating at 65° C., with wash in 0.2.X. SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to TRPM5 protein as encoded by SEQ ID NO:1, polymorphic variants, alleles, orthologs, and conservatively modified variants, or splice variants, or portions thereof, can be selected to obtain only those polyclonal antibodies that are specifically immunoreactive with TRPM5 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Expression of TRPM5 in Prokaryotes and Eukaryotes

To obtain high level expression of a cloned gene, such as those cDNAs encoding TRPM5, one typically subclones the TRPM5 gene into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al., supra. Bacterial expression systems for expressing the TRPM5 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the TRPM5-encoding nucleic acid in host cells. A typical expression cassette thus contains a promoter operably linked to the nucleic acid sequence encoding TRPM5 and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A.⁺, pMTO10/A.⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

Examples thereof include, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a TRPM5 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of TRPM5 protein, which can be purified therefrom using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing TRPM5.

After the expression vector is introduced into the cells, the transfected cells are cultured under conditions favoring expression of TRPM5, which may be recovered from the culture using standard techniques identified below.

Purification of TRPM5 Polypeptides

TRPM5 can be purified for, use in functional assays. Naturally occurring TRPM5 can be purified, e.g., from human tissue. Recombinant TRPM5 can be purified from any suitable expression system.

The TRPM5 protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant TRPM5 protein is being purified. For example, proteins having established molecular adhesion properties can be reversible fused to the TRPM5 protein. With the appropriate ligand, TRPM5 protein can be selectively adsorbed to a purification column and then freed from the column in a relatively pure form. The fused protein is then removed by enzymatic activity. Finally, TRPM5 protein could be purified using immunoaffinity columns.

Assays for Modulators of TRPM5 Protein

A. Assays

Modulation of a TRPM5 protein, can be assessed using a variety of in vitro and in vivo assays, including cell-based models as described herein. Such assays can be used to test for inhibitors and activators of TRPM5 protein, and, consequently, inhibitors and activators of specific taste modalities. Such modulators of TRPM5 protein are useful as flavor additives and flavor enhancers in foods, beverages, cosmetics, and medicaments and other consumer related products wherein taste is an issue. Modulators of TRPM5 protein are tested using either recombinant or naturally occurring TRPM5.

As noted above, preferably, the TRPM5 protein used in the subject cell based assays will be encoded by the modified human TRPM5 nucleic acid sequence contained in SEQ ID NO:2.

Measurement of TRPM5 activity, can be performed using a variety of assays, in vitro, in vivo, and ex vivo, as described herein. To identify molecules capable of modulating TRPM5, assays are performed to detect the effect of various candidate modulators on TRPM5 activity in a cell. Putative TRPM5 modulators can, in addition, be tested in human or animal taste tests to assess their affect on specific taste modalities such as bitter, swet or umami taste.

The channel activity of TRPM5 proteins can be assayed using a variety of assays to measure changes in ion fluxes including patch clamp techniques, measurement of whole cell currents, radiolabeled ion flux assays, or a flux assay coupled to atomic absorption spectroscopy and fluorescence assays using voltage-sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)). For example, a nucleic acid encoding a TRPM5 protein or homolog thereof can be injected into Xenopus oocytes. Channel activity can then be assessed by measuring changes in membrane polarization, i.e., changes in membrane potential. One preferred means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., Pflugers. Archiv. 391:185 (1981).

Preferably in the present invention the effect of compounds on TRPM5 activity is assessed by detecting changes in membrane potential using cells (HEK-293) that express TRPM5 and which preferably express a GPCR such as a taste GPCR and which are loaded with an ion specific dye or membrane potential dye, and membrane potential changes in response to TRPM5 modulators detected fluorimetrically, preferably by use of FLIPR.

However, the activity of TRPM5 polypeptides can be also assessed using a variety of other in vitro and in vivo assays to determine functional, chemical, and physical effects, e.g., measuring the binding of TRPM5 to other molecules, including peptides, small organic molecules, and lipids; measuring TRPM5 protein and/or RNA levels, or measuring other aspects of TRPM5 polypeptides, e.g., transcription levels, or physiological changes that affects TRPM5 activity. When the functional consequences are determined using intact cells or animals, one can also measure a variety of effects such as changes in cell growth or pH changes, cGMP, or cAMP, or components or regulators of the phospholipase C signaling pathway. Such assays can be used to test for both activators and inhibitors of KCNB proteins. Modulators thus identified are useful for, e.g., many diagnostic and therapeutic applications.

In Vitro Assays

Assays to identify compounds with TRPM5 modulating activity can be performed in vitro. Such assays can use full length TRPM5 protein or a variant thereof or a fragment of a TRPM5 protein, such as an extracellular domain or a cytoplasmic domain, optionally fused to a heterologous protein to form a chimera. In a preferred embodiment, the full-length polypeptide can be used in high throughput binding assays to identify compounds that modulate TRPM5 activity. Purified recombinant or naturally occurring TRPM5 protein can be used in the in vitro methods of the invention. In addition to purified TRPM5 protein or fragment thereof, the recombinant or naturally occurring TRPM5 protein can be part of a cellular lysate or a cell membrane. As described below, the binding assay can be either solid state or soluble. Preferably, the protein, fragment thereof or membrane is bound to a solid support, either covalently or non-covalently. Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the TRPM5 protein or fragment thereof is contacted with a potential modulator and incubated for a suitable amount of time. In one embodiment, the potential modulator is bound to a solid support, and the TRPM5 protein is added. In another embodiment, the TRPM5 protein is bound to a solid support. A wide variety of modulators can be used, as described below, including small organic molecules, peptides, antibodies, and TRPM5 ligand analogs. A wide variety of assays can be used to identify TRPM5-modulator binding, including labeled protein-protein binding assays, electrophoretic mobility shifts, immunoassays, enzymatic assays such as phosphorylation assays, and the like.

High throughput functional genomics assays can also be used to identify modulators of taste by identifying compounds that disrupt protein interactions between TRPM5 and other proteins to which it binds. Such assays can, e.g., monitor changes in cell surface marker expression, changes in intracellular calcium, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the peptide or with the protein encoded by the cDNA (e.g., TRPM5) can be isolated using a yeast two-hybrid system, mammalian two hybrid system, or phage display screen, etc. Targets so identified can be further used as bait in these assays to identify additional components that may interact with the TRPM5 channel which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Cell-Based In Vivo Assays

In another embodiment, TRPM5 protein can be expressed in a cell, and functional, e.g., physical and chemical or phenotypic, changes are assayed to identify TRPM5 modulators. Cells expressing TRPM5 proteins can also be used in binding assays. Any suitable functional effect can be measured, as described herein. For example, changes in membrane potential, changes in intracellular ion levels, and ligand binding are all suitable assays to identify potential modulators using a cell based system. Suitable cells for such cell based assays include both primary cells, e.g., taste cells that express a TRPM5 protein and cell lines. The TRPM5 protein can be naturally occurring or recombinant. Also, as described above, fragments of TRPM5 proteins or chimeras with ion channel activity can be used in cell based assays. For example, a transmembrane domain of a TRPM5 protein can be fused to a cytoplasmic domain of a heterologous protein, preferably a heterologous ion channel protein. Such a chimeric protein would have ion channel activity and could be used in cell based assays of the invention. In another embodiment, a domain of the TRPM5 protein, such as the extracellular or cytoplasmic domain, is used in the cell-based assays of the invention.

In another embodiment, cellular TRPM5 polypeptide levels are determined by measuring the level of protein or mRNA. The level of TRPM5 protein or proteins related to TRPM5 ion channel activation are measured using immunoassays such as western blotting, ELISA and the like with an antibody that selectively binds to the TRPM5 polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, TRPM5 expression can be measured using a reporter gene system. Such a system can be devised using a TRPM5 protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited TRPM5 will alter the properties of target enzymes, second messengers, channels, and other effector proteins. The examples include the activation of phospholipase C and other signaling systems. Downstream consequences can also be examined such as generation of diacyl glycerol and IP3 by phospholipase C.

Assays for TRPM5 activity include cells that are loaded with ion or voltage sensitive dyes to report receptor activity, e.g., by observing sodium influx. Assays for determining activity of such receptors can also use known agonists and antagonists for TRPM5 receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog.

Animal Models

Transgenic animal technology including gene knockout technology, for example as a result of homologous recombination with an appropriate gene targeting vector, or gene overexpression, will result in the absence or increased expression of the TRPM5 protein. The same technology can also be applied to make knock-out cells. When desired, tissue-specific expression or knockout of the TRPM5 protein may be necessary. Transgenic animals generated by such methods find use as animal models of tastant responses.

Knock-out cells and transgenic mice can be made by insertion of a marker gene or other heterologous gene into an endogenous TRPM5 gene site in the mouse genome via homologous recombination. Such mice can also be made by substituting an endogenous TRPM5 with a mutated version of the TRPM5 gene, or by mutating an endogenous TRPM5, e.g., by exposure to known mutagens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual (1988) and Teratocarcinomas and Embryonic Stem Cells: A Practical Approach (Robertson, ed., 1987).

B. Modulators

The compounds tested as modulators of TRPM5 protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of an TRPM5 protein. Typically, test compounds will be small organic molecules, peptides, lipids, and lipid analogs. In one embodiment, the compound is a menthol analog, either naturally occurring or synthetic.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md.).

C. Solid State and Soluble High Throughput Assays

In one embodiment the invention provides soluble assays using a TRPM5 protein, or a cell or tissue expressing a TRPM5 protein, either naturally occurring or recombinant. In another embodiment, the invention provides solid phase based in vitro assays in a high throughput format, where the TRPM5 protein or fragment thereof, such as the cytoplasmic domain, is attached to a solid phase substrate. Any one of the assays described herein can be adapted for high throughput screening, e.g., ligand binding, sodium flux, change in membrane potential, etc.

In the high throughput assays of the invention, either soluble or solid state, it is possible to screen up to several thousand different modulators or ligands in a single day. This methodology can be used for TRPM5 proteins in vitro, or for cell-based or membrane-based assays comprising an TRPM5 protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

Having described the invention supra, the following examples provide further illustration of some preferred embodiments of the invention. These examples are provided only for purposes of illustration and should not be construed as limiting the subject invention.

### EXAMPLES

Exemplary Materials and Methods Used for Calcium Imaging Experiments

HEK-293 cells (about 50-70% confluency) contained in 10 cm dishes are transfected with 5 μg of TRPM5 DNA and pcDNA3 and 2 μg of RFP plasmids using TransH293.

After 24 hours, the cells are split into 384-well plates at ˜50,000 cells/well.

At 48 hours post-transfection the cells are loaded with membrane potential dyes in HBSS for 60 minutes at room temperature.

Compounds plates are prepared in HBSS.

Materials and Methods Used for Selection of Stable Clones

HEK-293 cells transfected with TRPM5 nucleic acid sequence containing plasmid that comprises neo marker and stable cell clones are selected using neomycin.

Screened clones are screened using membrane potential dyes and FLIPR, for a GPCR-induced change in membrane potential.

### Materials and Methods for Patch Clamp Electrophysiological Assays

Culture dishes (35 mm) containing coverslips were seeded with 25,000 or 50,000 HEK cells stably transfected with TRPM5 and whole-cell patch clamp recordings were performed at 24 or 48 hrs post plating, respectively. All recordings were made with an Axopatch200B amplifier, digitized with a DIGIDATA 1322A, acquired with pCLAMP10.0 and analyzed with Clampfit 10.0 (Molecular Devices). Data was filtered at 5 kHz and sampled at 10 kHz. Series resistance compensation was set at 60%. Patch pipettes were pulled from borosilicate glass. The pipette solution contained (in mM): 130 CsCl, 10 NaCl, 1 MgCl₂ and 10 HEPES (pH 7.4). The bath solution contained (in mM): 150 NaCl, 2 KCl, 1.5 CaCl₂, 1 MgCl₂ and 10 HEPES (pH 7.4). After establishment of the whole-cell configuration TRPM5 currents were measured using voltage ramps spanning from −80 mV to +80 mV and administered at 1 s intervals. During the voltage ramp the holding potential was held at −80 mV for 50 ms, then ramped to +80 mV over the course of 80 ms and held at +80 mV for the final 50 ms. The holding potential between ramps was 0 mV. Low-resolution time-courses of the inward and outward currents were extracted from the ramps at −80 mV and +80 mV, respectively.

### Example 1

Construction of a Modified hTRPM5 Nucleic Acid Sequence According to the Invention

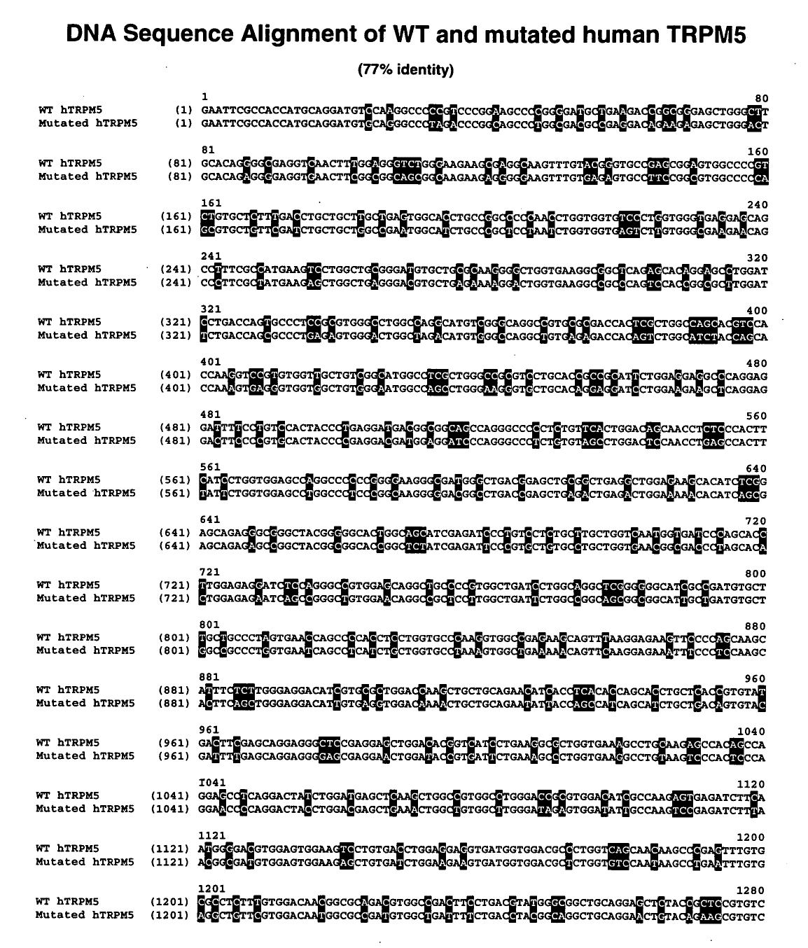

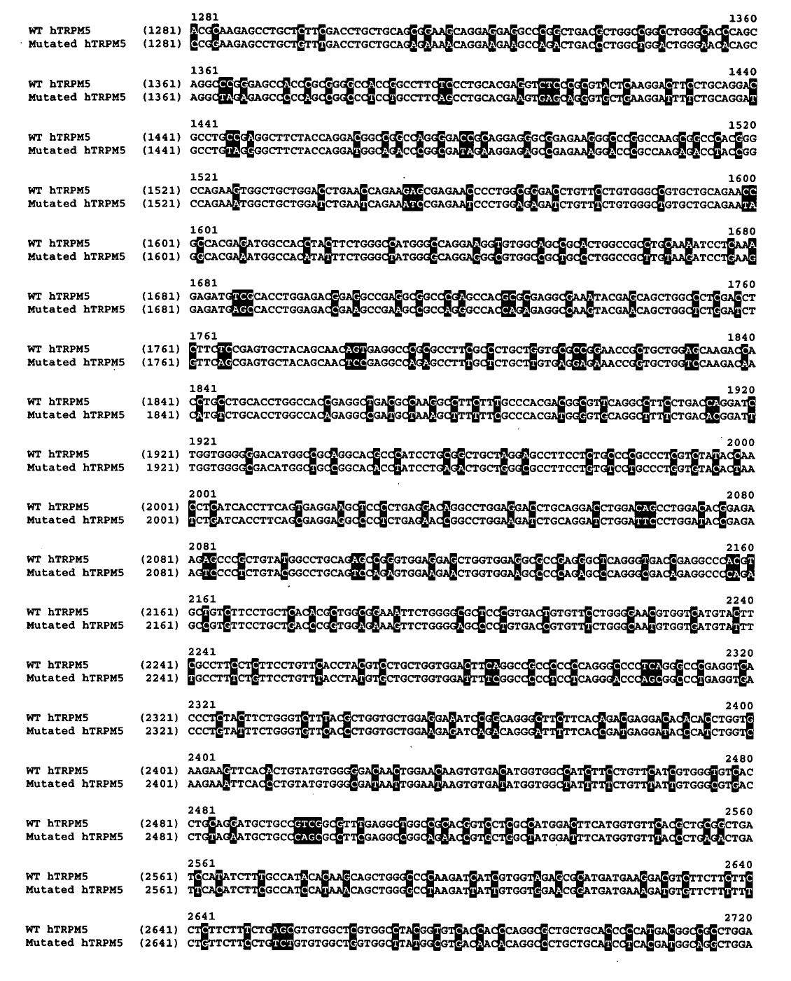

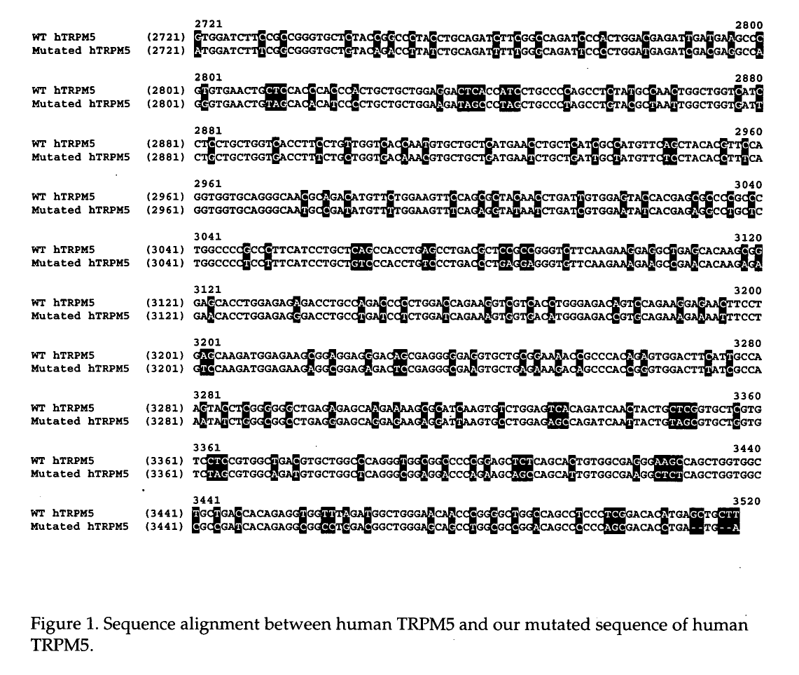

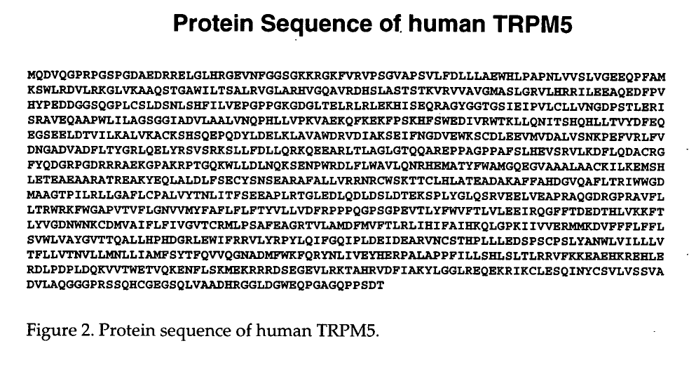

A modified human TRPM5 nucleic acid sequence was constructed using the native hTRPM5 sequence as a template. Specifically, in order to optimize expression of hTRPM5 in recombinant host cells (preferably human cells such as HEK-293 cells) silent mutations were introduced into the native hTRPM5 nucleic acid sequence as shown in FIG. 1 resulting in a modified sequence only possessing 77% sequence identity to the parent sequence. The mutations (shown in the alignment contained in FIG. 1) were made to remove putative TATA-boxes, chi-sites and ribosomal entry sites, AT-rich or GC-rich stretches, ARE, INS and CRS sequence elements and cryptic splice donor and acceptor sites.

These mutations did not change the amino acid sequence of the invention. When this sequence was expressed in HEK-293 cells and Xenopus oocytes (See examples below) it was found to result in a functional ion channel.

### Example 2

Human TRPM5 Responses to the Gαq-Coupled Receptor Agonist Carbachol.

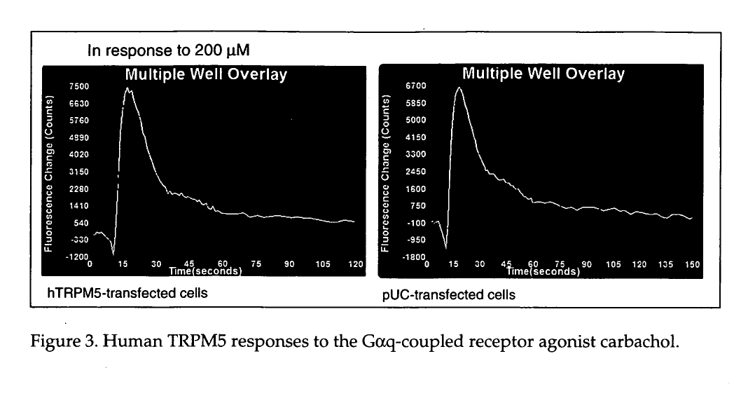

This experiment contained in FIG. 3 shows the effects of TRPM5 responses to the GPCR agonist carbachol. As shown in the Left Panel: HEK293 cells were transfected with a plasmid encoding hTRPM5. 48 hours later cells were loaded with either a membrane potential dye (Molecular Devices Corporation; R-8034 or DisBAC) or a Ca²⁺-sensitive dye (Fluo3). Activation of endogenously expressed muscarinic receptors with carbachol leads to Ca²⁺ mobilization (white trace). A concomitant increase in membrane potential is observed with the membrane potential dye signal (blue trace). As shown in the Right Panel: HEK293 cells were transfected with a control plasmid (pUC) and treated as described above. In the later experiment no change in membrane potential is observed although a significant increase in Ca²⁺ is triggered upon carbachol stimulation. Together these results show a TRPM5-dependent change in membrane potential in response to stimulation of a receptor-phospholipase C—Ca²⁺ pathway in HEK293 cells.

### Example 3

Human TRPM5 Responses to the Gαq-Coupled Receptor Agonists Carbachol, Angiotensin II and Histamine.

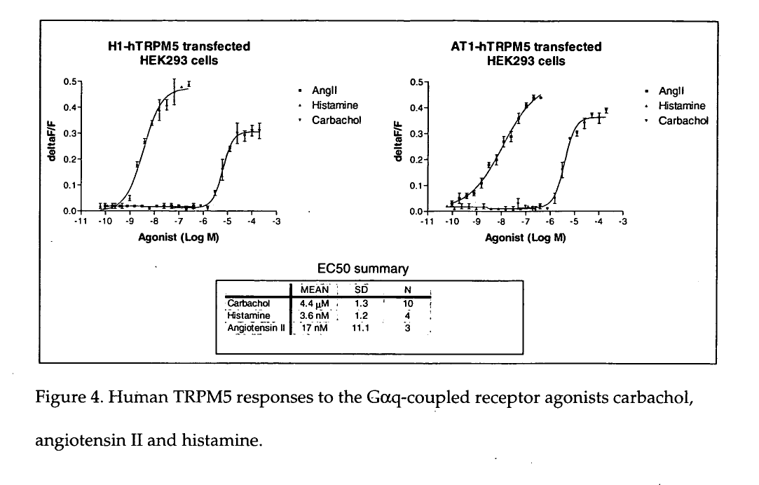

FIG. 4 contains another experiment showing human TRPM5 responses to the GPCR agonists carbachol, angiotensin II, and histamine. As shown in the Left Panel: HEK293 cells were transfected with plasmids encoding hTRPM5 and the H1 histamine receptor. 48 hours later cells were loaded with a membrane potential dye (R-8034; Molecular Devices Corporation). As shown therein, activation of transiently expressed H1 receptors with histamine as well as endogenously expressed muscarinic receptors with carbachol leads to a dose-dependent increase in membrane potential.

An unrelated agonist, angiotensin II (AngII) did not have any effect in this experiment. As shown in the Right Panel: HEK293 cells were transfected with plasmids encoding hTRPM5 and the AT1 angiotensin II receptor. 48 hours later cells were loaded with a membrane potential dye (R-8034; Molecular Devices Corporation). The experiments revealed that activation of transiently expressed AT1 receptors with AngII as well as endogenously expressed muscarinic receptors with carbachol led to a dose-dependent increase in membrane potential. In this experiment histamine has no effect since the cells were not transfected with the H1 receptor plasmid. In the two experiments the net increase in fluorescence measured after receptor stimulation was normalized to the initial fluorescence value measured before receptor stimulation (deltaF/F).

### Example 4

Receptor-Induced Changes in Membrane Potential are hTRPM5-Dependent.

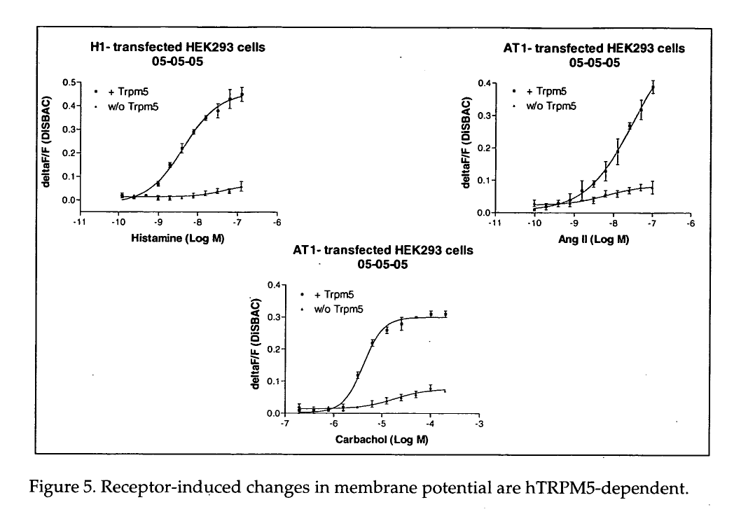

This experiment the results of which are shown in FIG. 5 evaluated TRPM5 induced changes in membrane potential. As shown in the left panel HEK293 cells were transfected with plasmids encoding the H1 histamine receptor with (red trace) or without (blue trace) a plasmid encoding hTRPM5. 48 hours later cells were loaded with a membrane potential dye (R-8034; Molecular Devices Corporation). Activation of transiently expressed H1 receptors with histamine led to a dose-dependent increase in membrane potential. In contrast no significant change in membrane potential is seen in cells lacking hTRPM5.

As shown in the right panel, HEK293 cells were transfected with plasmids encoding the AT1 angiotensin II receptor with (red trace) or without (blue trace) a plasmid encoding hTRPM5. 48 hours later cells were loaded with a membrane potential dye (R-8034; Molecular Devices Corporation). It was observed that the activation of transiently expressed AT1 receptors with angiotensin II (AngII) led to a dose-dependent increase in membrane potential. In contrast no significant change in membrane potential is seen in cells lacking hTRPM5.

As shown in the bottom panel, HEK293 cells were transfected with a plasmid encoding hTRPM5 (red trace) or a control plasmid (blue trace). 48 hours later cells were loaded with a membrane potential dye (R-8034; Molecular Devices Corporation). The results revealed that activation of endogenously expressed muscarinic receptors with carbachol led to a dose-dependent increase in membrane potential. In contrast no significant change in membrane potential is seen in cells lacking hTRPM5. In the three experiments the net increase in fluorescence measured after receptor stimulation was normalized to the initial fluorescence value measured before receptor stimulation (deltaF/F).

### Example 5

Gαq-Coupled Receptor-Induced Changes in Membrane Potential Require the Presence of Extracellular Na+.

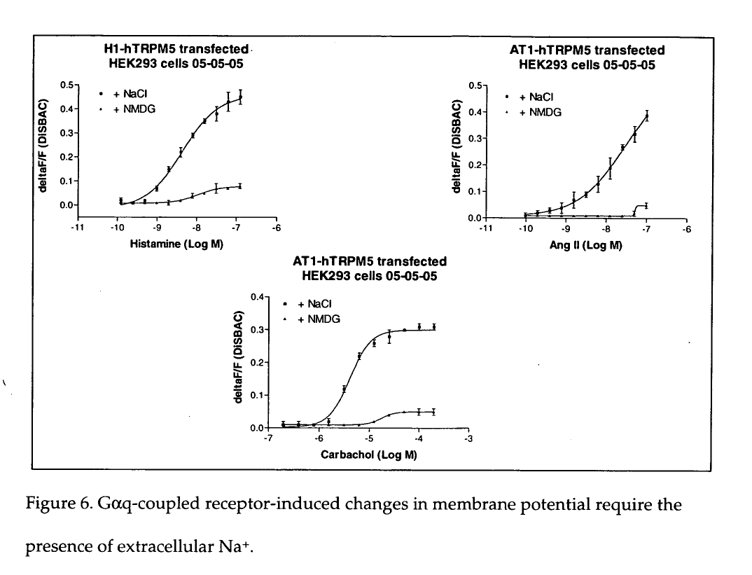

In this experiment contained in FIG. 6 it was shown that the changes in membrane potential elicited by a GPCR agonist require the presence of a monovalent cation such as sodium. As shown in the left panel, HEK293 cells were transfected with plasmids encoding the H1 histamine receptor and hTRPM5. 48 hours later cells were loaded with a membrane potential dye (R-8034; Molecular Devices Corporation). Cells were loaded and stimulated in the presence of NaCl (red trace) or NMDG as a Na⁺ replacement (blue trace). Histamine-induced changes in membrane potential were only observed in the presence of NaCl.

As shown in the right panel, HEK293 cells were transfected with plasmids encoding the AT1 angiotensin II receptor and hTRPM5. 48 hours later cells were loaded with a membrane potential dye (R-8034; Molecular Devices Corporation). Cells were loaded and stimulated in the presence of NaCl (red trace) or NMDG as a Na⁺ replacement (blue trace). AngII-induced changes in membrane potential were only observed in the presence of NaCl.

As shown in the bottom panel, HEK293 cells were transfected with plasmids encoding hTRPM5. 48 hours later cells were loaded with a membrane potential dye (R-8034; Molecular Devices Corporation). Cells were loaded and stimulated in the presence of NaCl (red trace) or NMDG as a Na⁺ replacement (blue trace). Carbachol-induced changes in membrane potential were only observed in the presence of NaCl. In the three experiments the net increase in fluorescence measured after receptor stimulation was normalized to the initial fluorescence value measured before receptor stimulation (deltaF/F).

### Example 6

Human TRPM5 Responses to the Ca2+ Ionophore Ionomycin.

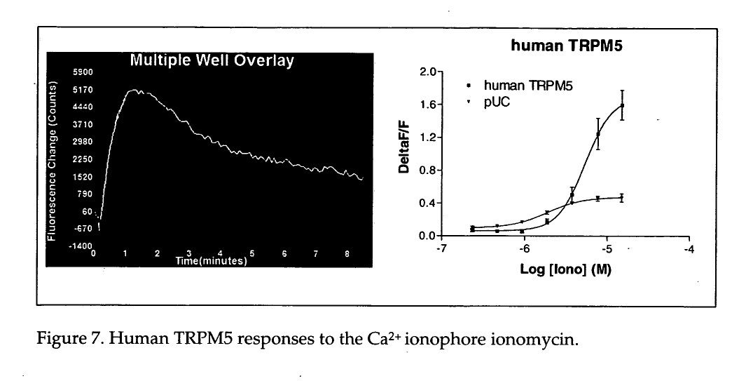

This experiment the results of which are contained in FIG. 7 compared TRPM5 responses to the Ca++ ionophore ionomycin. As shown in the left panel HEK293 cells were transfected with a plasmid encoding hTRPM5. 48 hours later cells were loaded with either a membrane potential dye (R-8034; Molecular Devices Corporation) or a Ca²⁺-sensitive dye (Fluo3). Application of 3 uM ionomycin leads to Ca²⁺ influx (blue trace). A concomitant increase in membrane potential was observed with the membrane potential dye signal (white trace).

As shown in the right panel HEK293 cells were transfected either with hTRPM5 or with a control plasmid (pUC) and loaded as described above. It was observed that ionomycin leads to a dose-dependent increase in membrane potential that is noticeably greater in hTRPM5-expressing cells.

### Example 7

Mouse TRPM5 Responses to the Ca2+ Ionophore Ionomycin and G□q-Coupled Receptor Agonists.

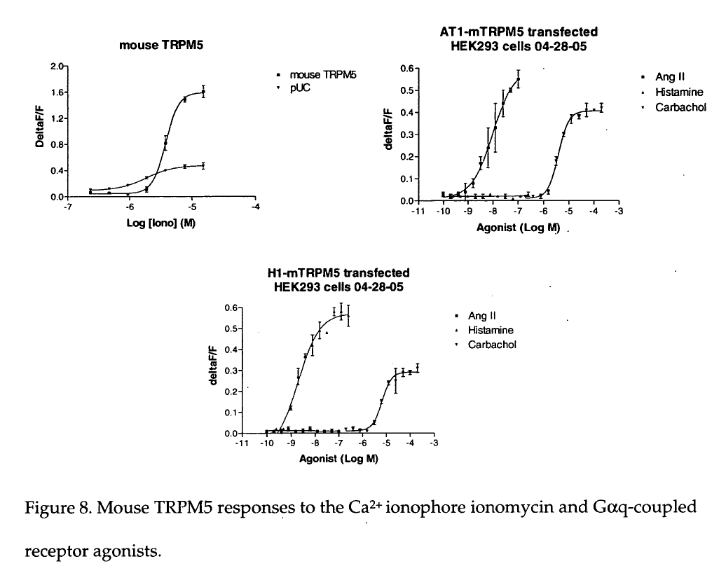

This experiment contained in FIG. 8 compares mTRPM5 responses to the calcium ionophore ionomycin and various GPCR agonists. As shown in the left top panel HEK293 cells were transfected with a plasmid encoding mouse TRPM5 (red trace) or a control plasmid (pUC; blue trace). 48 hours later cells were loaded with a membrane potential dye (R-8034; Molecular Devices Corporation). Application of ionomycin led to a dose-dependent increase in membrane potential that is significantly greater in mouse-TRPM5 transfected cells relative to pUC-transfected cells.

Further as shown in the right top panel HEK293 cells were transfected with plasmids encoding mouse TRPM5 and the angiotensin II AT1 receptor. 48 hours later cells were loaded with a membrane potential dye (R-8034; Molecular Devices Corporation). Activation of transiently expressed AT1 receptors with AngII as well as endogenously expressed muscarinic receptors with carbachol led to a dose-dependent increase in membrane potential. An unrelated agonist, histamine did not have any effect in this experiment.

As shown in the bottom panel HEK293 cells were transfected with plasmids encoding mouse TRPM5 and the H1 histamine receptor. 48 hours later cells were loaded with a membrane potential dye (R-8034; Molecular Devices Corporation). Activation of transiently expressed H1 receptors with histamine as well as endogenously expressed muscarinic receptors with carbachol led to a dose-dependent increase in membrane potential. In this experiment AngII had no effect since the cells were not transfected with the AT1 receptor plasmid. In the three experiments the net increase in fluorescence measured after receptor stimulation was normalized to the initial fluorescence value measured before receptor stimulation (deltaF/F).

### Example 8

hTRPM5 and Mouse TRPM5 Responses to a G□15-Coupled Receptor Agonist.

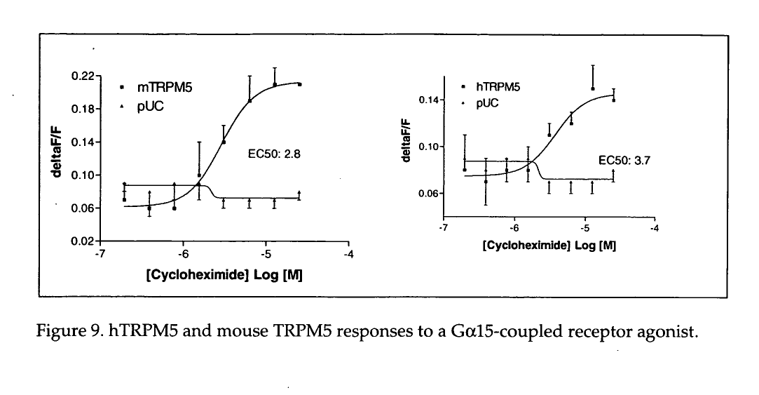

This experiment contained in FIG. 9 compares hTRPM5 and mTRPM5 responses to GPCR agonist compounds. As shown in the left panel HEK293 cells stably expressing the cycloheximide bitter receptor mT2R5 and the promiscuous G protein G□15 were transfected with a plasmid encoding for mouse TRPM05 or a control plasmid (pUC). 48 hours later cells were loaded with a membrane potential dye (R-8034; Molecular Devices Corporation). Cycloheximide induced a dose-dependent increase in membrane potential that was much greater than what is seen in cells not expressing mouse TRPM5.

As shown in the right panel HEK293 cells stably expressing the cycloheximide bitter receptor mT2R05 and the promiscuous G protein G□15 were transfected with a plasmid encoding for human TRPM5 or a control plasmid (pUC). 48 hours later cells were loaded with a membrane potential dye (R-8034; Molecular Devices Corporation). It was observed that cycloheximide induced a dose-dependent increase in membrane potential that is much greater than what is seen in cells not expressing human TRPM5.

### Example 9

hTRPM5 Response in a Stable Clone.

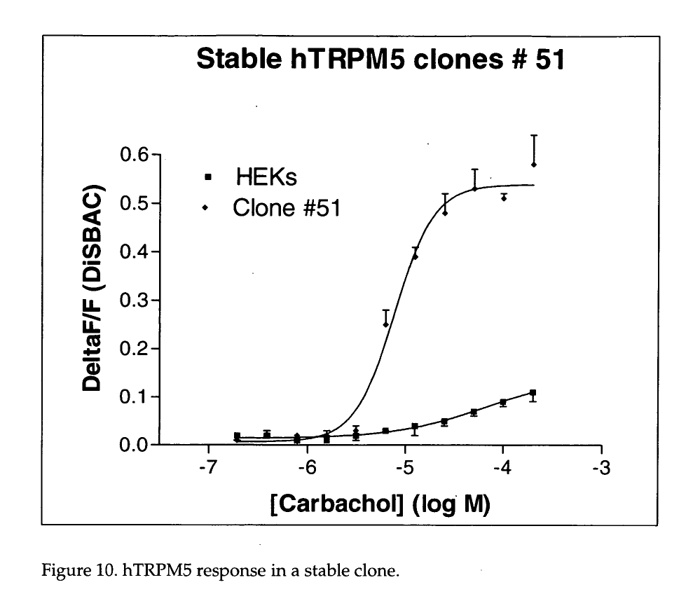

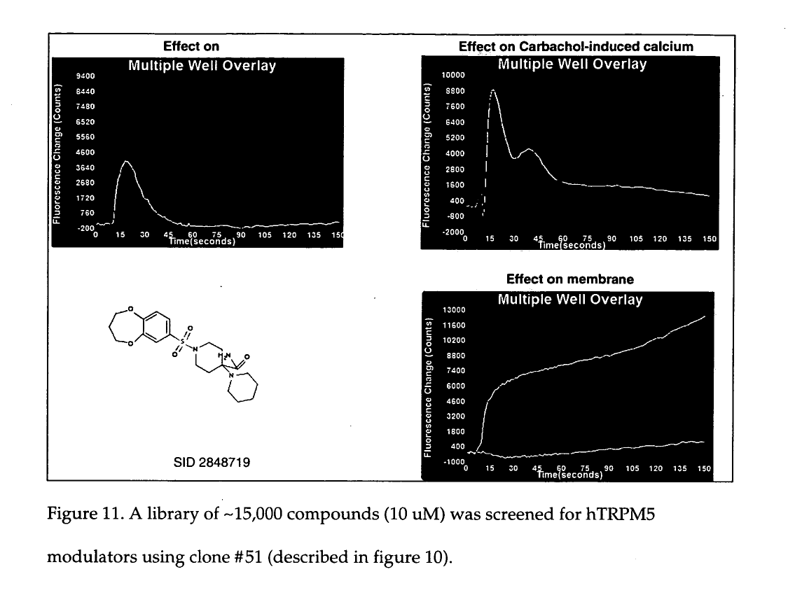

This experiment is contained in FIG. 10. In the experiment HEK293 cells were transfected with a plasmid encoding hTRPM5 in pcDNA3.Neo. 48 hours later cells were put in selection media (500 ug/ml neomycin) and stable clones were selected and expanded. Clone #51 was further characterized for carbachol-induced hTRPM5 activation. Cells were loaded with a membrane potential dye (R-8034; Molecular Devices Corporation). It was observed that carbachol induced a dose-dependent increase in membrane potential that is much greater than what is seen in cells not expressing hTRPM5 (HEKs).

### Example 10

Library Screening Results.

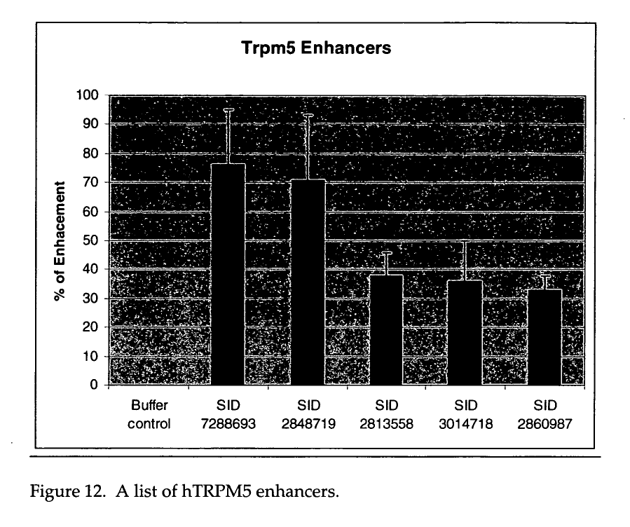

In this experiment the results of which are contained in FIG. 11 a library of ˜15,000 compounds (10 uM) was screened for hTRPM5 modulators using clone #51 (described in FIG. 10). In our initial screening conditions hTRPM5 was maximally stimulated with carbachol, using concentrations of 50 uM to 100 uM. Under these conditions some compounds, including SID 2848719 (blue traces in all panels), enhanced the carbachol-induced change in membrane potential (top left panel). These compounds did not enhance the carbachol-induced Ca²⁺ mobilization (top right panel) and they did not change the membrane potential in the absence of hTRPM5 (bottom right panel). Together, these results suggest that SID 2848719 and other compounds with similar profiles (see FIG. 12) boost the carbachol-induced change in membrane potential by enhancing hTRPM5 function.

### Example 11

Identified hTRPM5 Enhancers.

As shown in FIG. 12, five TRPM5 enhancer compounds were discovered during the screen described in FIG. 11. Each one of them displays profiles similar to another proprietary TRPM5 SID 2848719. They reproducibly boost the carbachol-induced change in membrane potential mediated by hTRPM5 without affecting the carbachol-induced Ca²⁺ mobilization or without changing the membrane potential on their own. These 5 compounds reproducibly enhance hTRPM5 by 33% to 75% at 10 uM. The depicted data correspond to the average±SD of a triplicates determination.

### Example 12

hTRPM5 Enhancers Improve the Potency and Efficacy of the Carbachol-Induced Change in Membrane Potential.

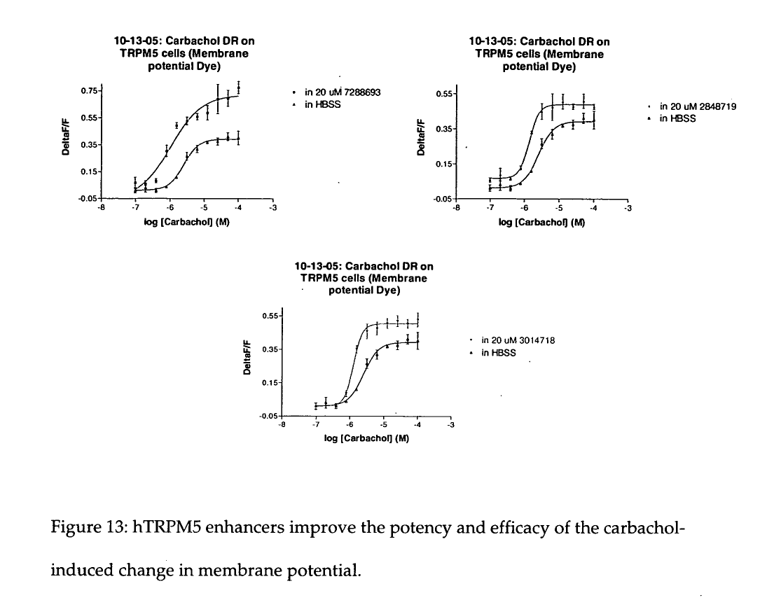

This example relates to the experiment depicted in FIG. 13. In this experiment the hTRPM5 stable cell line described in FIG. 10 was stimulated with increasing concentrations of the muscarinic receptor agonist carbachol in the presence or absence of 20 □M SIDs 7288693, SIDs 2848719 and SIDs 3014718. These compounds were observed to significantly increase the potency and efficacy of the carbachol-induced-TRPM5 mediated change in membrane potential.

### Example 13

hTRPM5 Enhancers Improve the Potency and Efficacy of the Ionomycin-Induced Change in Membrane Potential.

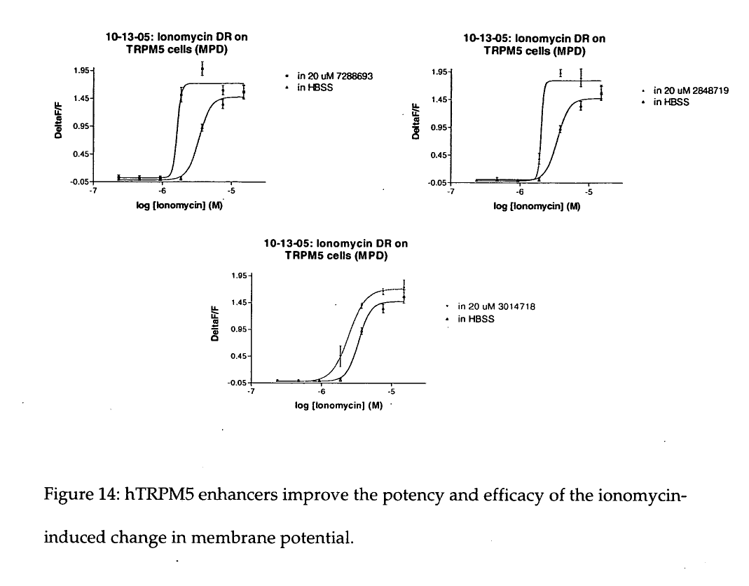

This example relates to the experiment depicted in FIG. 14. In this experiment the hTRPM5 stable cell line described in FIG. 10 was stimulated with increasing concentrations of the ionophore ionomycin in the presence or absence of 200M SIDs 7288693, SIDs 2848719 and SIDs 3014718. These compounds were found to significantly increase the potency and efficacy of the ionomycin-induced-TRPM5 mediated change in membrane potential.

### Example 14

Enhancement Properties of SID 7288693 at Different Levels of hTRPM5 Activity.

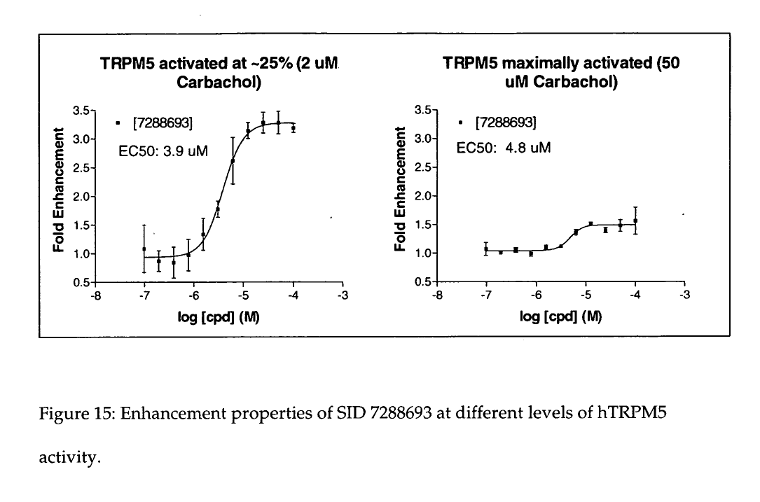

This example relates to the experiment contained in FIG. 15. In this experiment the hTRPM5 stable cell line described in FIG. 10 was stimulated with increasing concentrations of SID 7288693 in the presence of 2 □M carbachol (Left Panel) or 50 uM carbachol (Right Panel). It was observed that the enhancement effect (potency and efficacy) of SID 7288693 is vastly improved at 2 um carbachol a concentration producing about 10-25% of hTRPM5 activity.

### Example 15

Enhancement Properties of SID 2848719 at Different Levels of hTRPM5 Activity.

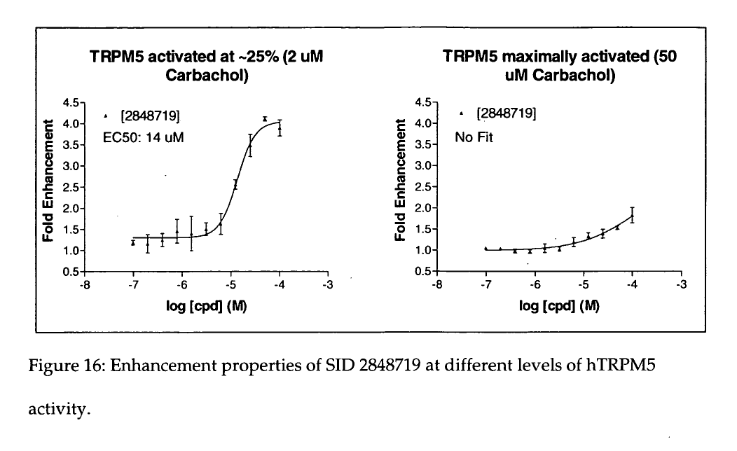

This example relates to the experiment contained in FIG. 16. In this experiment the hTRPM5 stable cell line described in FIG. 10 was stimulated with increasing concentrations of SID 2848719 in the presence of 20M carbachol (Left Panel) or 50 uM carbachol (Right Panel). It was observed that enhancement effect (potency and efficacy) of SID 2848719 is dramatically improved at 2 um carbachol, a concentration producing about 10-25% of hTRPM5 activity.

### Example 16

Enhancement Properties of SID 3014718 at Different Levels of hTRPM5 Activity.

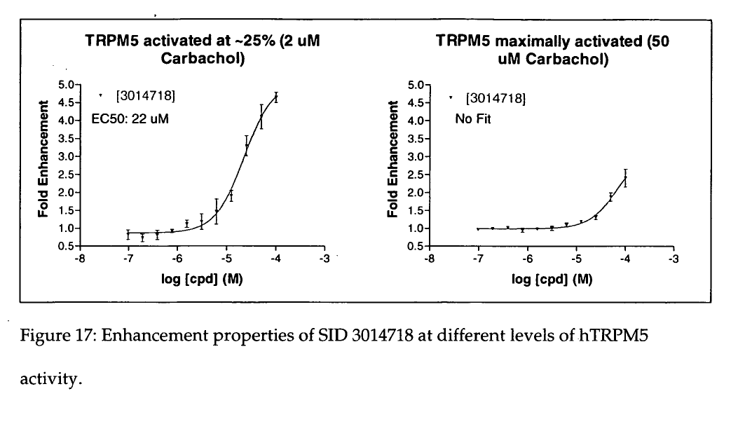

This example relates to the experiment contained in FIG. 17. In this experiment the hTRPM5 stable cell line described in FIG. 10 was stimulated with increasing concentrations of SID 3014718 in the presence of 2 □M carbachol (Left Panel) or 50 uM carbachol (Right Panel). Again it was observed that the enhancement effect (potency and efficacy) of SID 3014718 is dramatically improved at 2 um carbachol, a concentration producing about 10-25% of hTRPM5 activity.

### Example 17

Wild Type and Mutated TRPM5 Sequences Produce Identical Results in the FLIPR Assay.

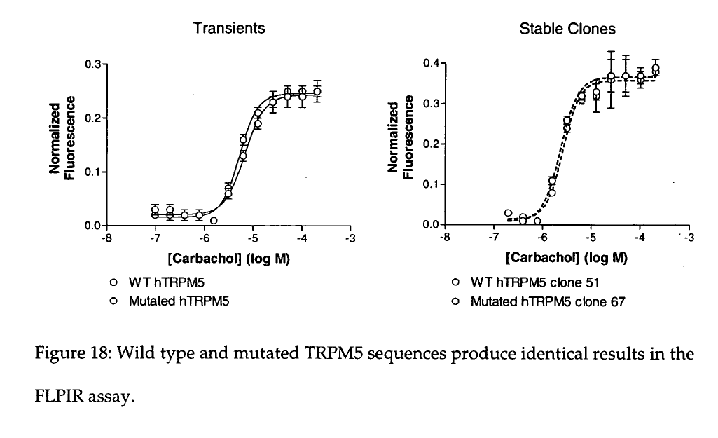

This example relates to the FLIPR assays contained in FIG. 18. As shown therein HEK-293 cells that express the mutant hTRPM5 nucleic acid sequence yielded about the same results as HEK-293 cells that express the wild-type sequence in both stable and transient clones contacted with carbachol.

### Example 18

Activity Distribution of 100,000 Screened in the TRPM5 Assay.

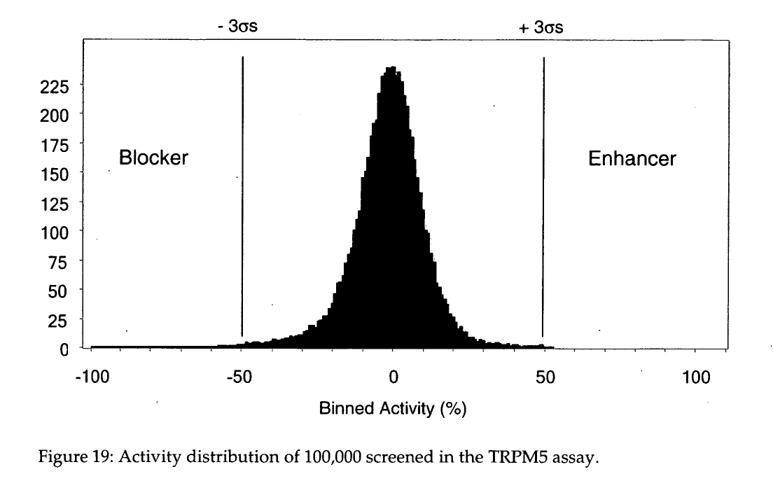

In this example shown in FIG. 19, more than 200,000 compounds (100,000 shown) were screened using conditions described in FIGS. 15, 16 and 17 and the mutated TRPM5 stable clone described in FIG. 18. These assay conditions allowed the identification of both enhancers and blockers of TRPM5.

### Example 19

Novel SID 15776016 Enhancer

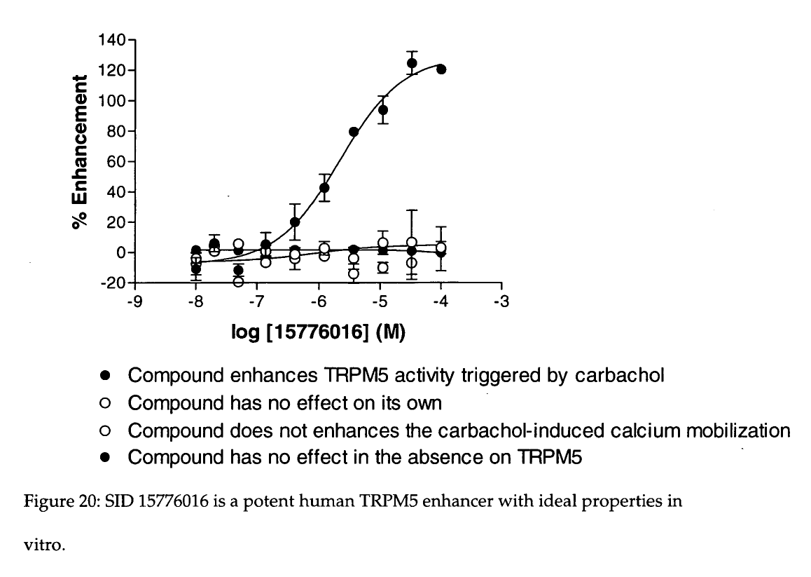

As shown in the experimental data in FIG. 20, SID 15776016 is a potent human TRPM5 enhancer with ideal properties in vitro. This compound SID 15776016 was identified as a TRPM5 enhancer in the screen described in FIG. 19. As shown therein, this compound is a relatively potent enhancer (EC50˜2 uM) and it does not display any non-specific side effect in the assay.

### Example 20

Confirmation of SID 15776016 Enhancement Properties in Patch Clamp Experiments.

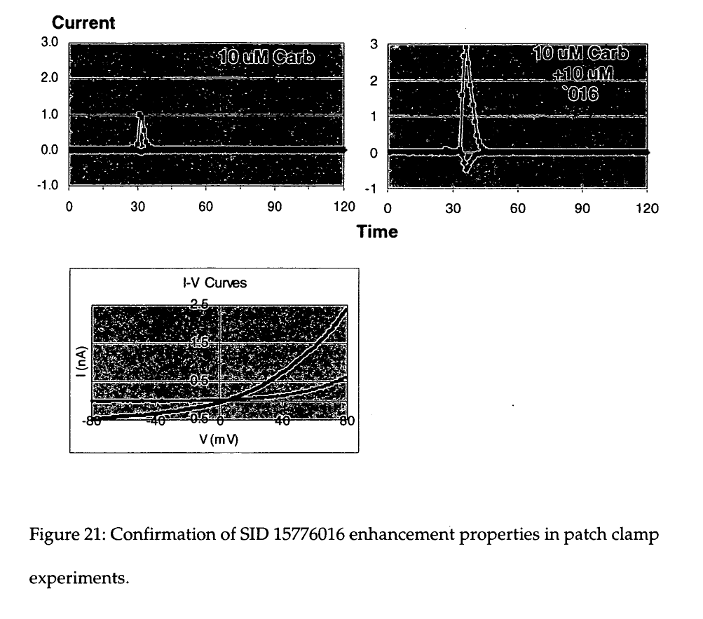

In this experiment contained in FIG. 21, the stable cell line expressing human TRPM5 described in FIG. 18 was patch clamped (whole cell configuration) and currents were recorded during a voltage ramp from −80 mV to +80 mV. In the Figure the top panels (left and right) show that SID 15776016 significantly increases the carbachol-induced current (by about 2 and 11 fold for the outward and inward currents, respectively). The bottom panel in the Figure shows the I-V relationship in the presence of enhancer (and carbachol; red line) or in the absence of enhancer (carbachol only, blue line).

### Example 21

Determination of Enhancement Properties for SID 15776016 in a Taste Test.

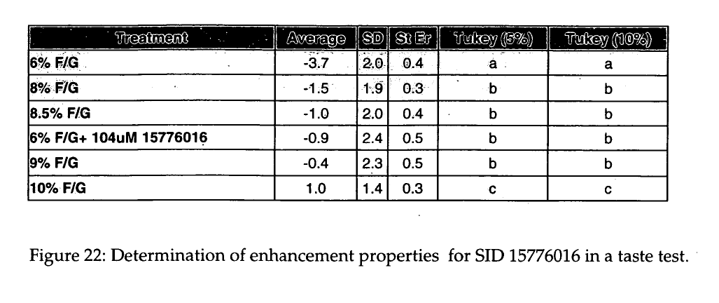

In this experiment contained in FIG. 22, average sweetness scores, n=22 (11 Panelists×2 reps) were determined for the identified enhancer compound. Significant differences were calculated using Tukey's HSD (5% risk level). Tukey's (5%)=1.352. Samples with the same Tukey's lettering are not significantly different from each other. All samples were prepared in Low Sodium Buffer (LSB) and 0.1% ethanol.

As shown therein, test sample 104 uM '016 in a solution of 6% Fructose-Glucose (F/G) had a greater sweetness intensity than the standard solution of 6% Fructose-Glucose and had a similar sweetness intensity to the 8%, 8.5% and 9% standard solutions of Fructose-Glucose.

### Example 22

SID 12038967 is a Potent Human TRPM5 Blocker with Ideal Properties In Vitro.

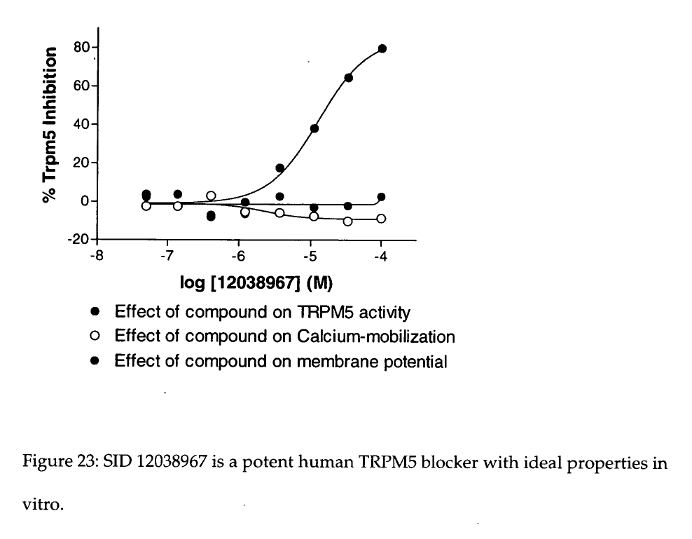

In this experiment contained in FIG. 23, the proprietary blocker compound SID 12038967 was identified as a TRPM5 blocker in the screen described in FIG. 19. This compound was observed to be a relatively potent blocker (EC50˜12 uM) and to not display any non-specific side effect in the assay.

We claim:

1. A screening assay for identifying compounds that modulate taste comprising:
(i) contacting a cell that stably or transiently expresses a functional human or rodent TRPM5 ion channel and further optionally expresses a G protein coupled receptor (GPCR) involved in taste with at least one putative taste modulatory compound;
(ii) assaying whether said compound results in a detectable change in TRPM5 activity; and
(iii) identifying said compound as one that putatively modulates taste based on whether it affects TRPM5 activity;
(iv) confirming in a taste test whether compound modulates taste.

2. The assay of claim 1 wherein the cell expresses at least one T1R or T2R.

3. The assay of claim 2 wherein the cell expresses a human or rodent T2R.

4. The assay of claim 2 wherein the cell expresses human or rodent T1R1 and T1R3.

5. The assay of claim 2 wherein said cell expresses human or rodent T1R2 and T1R3.

6. The assay of any one of claims 1-5 wherein the effect of said compound on TRPM5 function is detected by assaying for changes in membrane potential.

7. The assay of claim 1 wherein said cell expresses a GPCR introduced by recombinant means.

8. The assay of claim 1 which includes contacting said cell with an ionophore.

9. The assay of claim 8 wherein said ionophore is ionomycin or A-23187.

10. The assay of claim 1 wherein changes in TRPM5 activity are detected fluorimetrically.

11. The assay of claim 10 wherein said cell is loaded with a membrane potential dye or an ion sensitive dye.

12. The assay of claim 1 wherein the effect of said compound on TRPM5 activity is detected electrophysiologically.

13. The assay of claim 12 which comprises a patch clamp assay.

14. The assay of claim 12 which comprises a voltage clamp assay.

15. The assay of claim 1 wherein said cell is a mammalian cell, amphibian cell, bacterial cell, yeast cell, or an oocyte.

16. The assay of claim 15 wherein the mammalian cell is selected from a HEK-293 cell, CHO cell, BHK cell, MDK cell, monkey L cell, African Green monkey cell, or COS cell.

17. The assay of claim 11 wherein the ion sensitive dye is a sodium, lithium, potassium or cesium ion sensitive dye.

18. The assay of claim 18 wherein the dye comprises CBFI, PBFI, R-8034 or DisBAC.

19. The assay of any one of claims 1-5 wherein the cell is contacted wih a known activator of a GPCR expressed by said cell.

20. The assay of claim 19 wherein said compound is a known activator of a T2R or T1R expressed by said cell.

21. The assay of claim 1 which detects the effect of said compound on TRPM5 activity using a radiolabeled or non-radiolabeled ion flux assay.

22. The assay of claim 21 which comprises a radiolabeled Na⁺ flux assay or a non-radiolabeled Li+ or Rb+flux assay coupled with atomic absorption spectroscopy.

23. The assay of claim 1 which additionally contacts said cell with a compound that stimulates a receptor-phospholipase Ca++ pathway in said cell.

24. The assay of claim 1 which is a high throughput screening assay.

25. The assay of claim 1 wherein said cell expresses a Gq or promiscuous G protein.

26. The assay of claim 1 wherein the effect of said compound on TRPM5 activity is detected using a fluorimetric imaging assay.

27. The assay if claim 26 which uses an automated imaging device.

28. The assay of claim 27 wherein said device is a fluorescence plate reader.

29. The assay of claim 1 wherein changes in TRPM5 activity are detected using a voltage imaging plate reader.

30. The assay of claim 28 which uses a calcium sensitive dye.

31. The assay of claim 1 which includes the addition of a GPCR activator that is added at sub-optimal concentrations.

32. The assay of claim 31 wherein the activator is added at a dosage that results in no more than about 25% maximal activation of said GPCR.

33. The assay of claim 32 wherein said compound is a T1R or T2R activator.

34. The assay of claim 33 wherein said compound is a bitter compound that activates a T2R expressed by said cell.

35. The assay of claim 33 wherein said compound is a sweet or umami compound that activates a T1R expressed by said cell.

36. The assay of claim 1 wherein said cell is a HEK-293 cell that expresses a human TRPM5 and a taste GPCR.

37. The assay of claim 1 wherein said cell expresses a modified human TRPM5 nucleic acid sequence.

38. The assay of claim 37 wherein said modified sequence (i) comprises a nucleic acid sequence which is modified relative to the human TRPM5 nucleic acid sequence contained in SEQ ID NO: 2 or another wild-type TRPM5 noted acid sequence at least by the introduction of mutations selected from the group consisting of removal of putative (1) TATA-boxes, (2) chi-sites, (3) ribosomal entry sites, (4) ARE, INS or CRS sequence elements, and (5) cryptic splice donor and acceptor sites, and (ii) is expressed in human cells as an active ion channel.
